(19) United States
(12) Patent Application Publication
(10) Pub. No.: US 2007/0299445 A1
(43) Pub. Date: Dec. 27, 2007

(54) SPINE TREATMENT DEVICES AND METHODS

(76) Inventors: John H. Shadduck, Tiburon, CA (US); Csaba Truckai, Saratoga, CA (US); Robert Luzzi, Pleasanton, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/767,402

(22) Filed: Jun. 22, 2007

Related U.S. Application Data
(60) Provisional application No. 60/934,428, filed on Jun. 22, 2006.

(51) Int. Cl. A61B 17/58

(52) U.S. Cl. 606/61; 606/100; 606/73

(57) ABSTRACT

A modular implant system and method for dynamic stabilization of a spine segment that can be implanted in a minimally invasive posterior approach. In one embodiment, the implant device has an inferior body portion configured for fixation in a sacrum or ilium and a superior body portion configured for engaging one or more spinous processes to limit extension and optionally flexion while off-loading a spine segment. The implant system can be configured to stabilize a spine segment, re-distribute loads with the spine segment and still allow spine lateral bending and torsion. Besides being far less invasive than other procedures, the system and method is reversible and adjustable post-implant.

### CROSS-REFERENCE TO RELATED APPLICATIONS

This application claims the benefit of Provisional U.S. Patent Application No. 60/934,428 filed Jun. 22, 2006 (Attorney Docket No. S-7700-380), the entire contents of which are hereby incorporated by reference and should be considered a part of this specification.

### BACKGROUND OF THE INVENTION

1. Field of the Invention

The invention relates generally to implant systems and methods for treating a spine disorder, and more particularly relates to minimally invasive implant devices and systems that are configured for re-distributing loads within a spine segment while still allowing for flexion, extension, lateral bending and torsion.

2. Description of the Related Art

Thoracic and lumbar spinal disorders and discogenic pain are major socio-economic concerns in the United States affecting over 70% of the population at some point in life. Low back pain is the most common musculoskeletal complaint requiring medical attention; it is the fifth most common reason for all physician visits. The annual prevalence of low back pain ranges from 15% to 45% and is the most common activity-limiting disorder in persons under the age of 45.

Degenerative changes in the intervertebral disc often play a role in the etiology of low back pain. Many surgical and non-surgical treatments exist for patients with degenerative disc disease (DDD), but often the outcome and efficacy of these treatments are uncertain. In current practice, when a patient has intractable back pain, the physician's first approach is conservative treatment with the use of pain killing pharmacological agents, bed rest and limiting spinal segment motion. Only after an extended period of conservative treatment will the physician consider a surgical solution, which often is spinal fusion of the painful vertebral motion segment. Fusion procedures are highly invasive procedure that carries surgical risk as well as the risk of transition syndrome described above wherein adjacent levels will be at increased risk for facet and discogenic pain.

More than 150,000 lumbar and nearly 200,000 cervical spinal fusions are performed each year to treat common spinal conditions such as degenerative disc disease and spondylolisthesis, or misaligned vertebrae. Some 28 percent are multi-level, meaning that two or three vertebrae are fused. Such fusions “weld” unstable vertebrae together to eliminate pain caused by their movement. While there have been significant advances in spinal fusion devices and surgical techniques, the procedure does not always work reliably. In one survey, the average clinical success rate for pain reduction was about 75%; and long time intervals were required for healing and recuperation (3-24 months, average 15 months). Probably the most significant drawback of spinal fusion is termed the “transition syndrome” which describes the premature degeneration of discs at adjacent levels of the spine. This is certainly the most vexing problem facing relatively young patients when considering spinal fusion surgery.

Many spine experts consider the facet joints to be the most common source of spinal pain. Each vertebra possesses two sets of facet joints, one set for articulating to the vertebra above and one set for the articulation to the vertebra below. In association with the intervertebral discs, the facet joints allow for movement between the vertebrae of the spine. The facet joints are under a constant load from the weight of the body and are involved in guiding general motion and preventing extreme motions in the trunk. Repetitive or excessive trunkal motions, especially in rotation or extension, can irritate and injure facet joints or their encasing fibers. Also, abnormal spinal biomechanics and bad posture can significantly increase stresses and thus accelerate wear and tear on the facet joints.

Recently, technologies have been proposed or developed for disc replacement that may replace, in part, the role of spinal fusion. The principal advantage proposed by complete artificial discs is that vertebral motion segments will retain some degree of motion at the disc space that otherwise would be immobilized in more conventional spinal fusion techniques. Artificial facet joints are also being developed. Many of these technologies are in clinical trials. However, such disc replacement procedures are still highly invasive procedures, which require an anterior surgical approach through the abdomen.

Clinical stability in the spine can be defined as the ability of the spine under physiologic loads to limit patterns of displacement so as to not damage or irritate the spinal cord or nerve roots. In addition, such clinical stability will prevent incapacitating deformities or pain due to later spine structural changes. Any disruption of the components that stabilize a vertebral segment (e.g., disc, facets, and ligaments) decreases the clinical stability of the spine.

Improved devices and methods are needed for treating dysfunctional intervertebral discs and facet joints to provide clinical stability, in particular: (i) implantable devices that can be introduced to offset vertebral loading to treat disc degenerative disease and facets through least invasive procedures; (ii) implants and systems that can restore disc height and foraminal spacing; and (iii) implants and systems that can re-distribute loads in spine flexion, extension, lateral bending and torsion.

### SUMMARY OF THE INVENTION

In accordance with one embodiment, a spine treatment device is provided. The spine treatment device comprises an implant body extending from a first body portion to a second body portion, the first body portion comprising a pair of elongated extending members configured for fixation to a sacral or iliac bone of a spine, the second body portion comprising at least one connecting member extending transversely between the pair of extending members, the at least one connecting member configured to contact a surface of a spinous process of a spine segment to limit at least one of extension and flexion of the spine segment.

In accordance with another embodiment, an implant for treatment of a spine segment is provided. The implant comprises a pair legs coupleable to a sacral o iliac bone of a spine, and first and second connecting members extending between the pair of legs and positionable on either side of a spinous process such that one of the connecting members contacts an inferior surface of the spinous process and another of the connecting members contacts a superior surface of the spinous process so as to limit extension and flexion of the spine segment.

In accordance with yet another embodiment, a method for treating an abnormal spine segment is provided. The method comprises fixating a first body portion of a stabilization device to at least one of a sacral bone and an iliac bone, and positioning a second body portion of the stabilization device in contact with a spinous process of a vertebra, wherein the stabilization device varies a load-carrying characteristic of the spine segment.

### BRIEF DESCRIPTION OF THE DRAWINGS

These and other features, aspects and advantages of the present inventions will now be described in connection with preferred embodiments, in reference to the accompanying drawings. The illustrated embodiments, however, are merely examples and are not intended to limit the inventions. The drawings include the following 14 figures, wherein:

FIG. 1 is a schematic posterior view of a patient's lumbar spine, sacrum and ilium with a spine implant device, in accordance with one embodiment.

FIG. 2 is a schematic side view of a spine and sacrum showing the implant device of FIG. 1.

FIG. 3A is a schematic plan view of the spine implant of FIG. 1.

FIG. 3B is a schematic side view of the spine implant of FIG. 1.

FIG. 4 is a schematic plan view of another embodiment of a spine implant similar to that of FIG. 1.

FIG. 5 is a schematic plan view of another embodiment of a spine implant similar to that of FIG. 1.

FIG. 6 is a schematic posterior view of the implant device of FIG. 5 after implantation in a patient's lumbar spine.

FIG. 7 is a schematic side view of the implant device of FIG. 6.

FIGS. 8 and 9 are schematic posterior and side views of an implant device as in FIG. 5 with the inferior implant body fixated in the patient's ilium.

FIG. 10 is a schematic posterior view of another embodiment of a spinal implant device similar to that of FIG. 5 implanted so that superior elements of the device engage both inferior and superior surfaces of a spinous process to limit extension and flexion and off-load the spine segment.

FIG. 11 is a schematic posterior view of another embodiment of a spinal implant device similar to that of FIG. 10 implanted so that superior elements of the device engage a plurality of spinous processes to limit extension and flexion therein and off-load the spine segment.

FIG. 12 is a schematic posterior view of another embodiment of a spinal implant device similar to that of FIG. 11 implanted so that superior elements of the device engage a plurality of spinous processes but with independent elements coupled to sacral and/or iliac bones.

FIG. 13 is a schematic posterior view of another embodiment of a spinal implant device.

### DETAILED DESCRIPTION OF THE PREFERRED EMBODIMENT

Various embodiments of spine implant devices are disclosed herein that have similar components. As such, identical reference numerals are used to identify similar components of the different spine implant device embodiments.

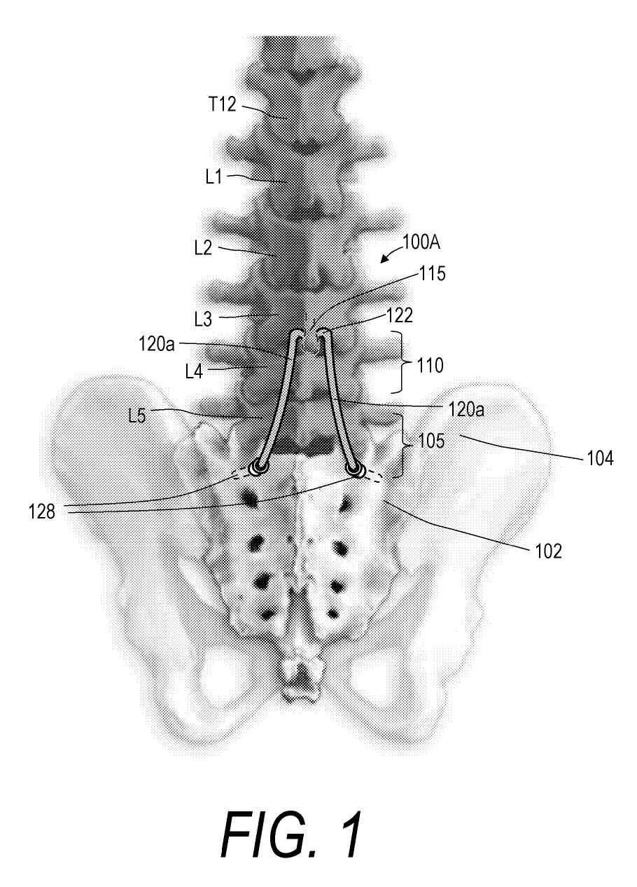

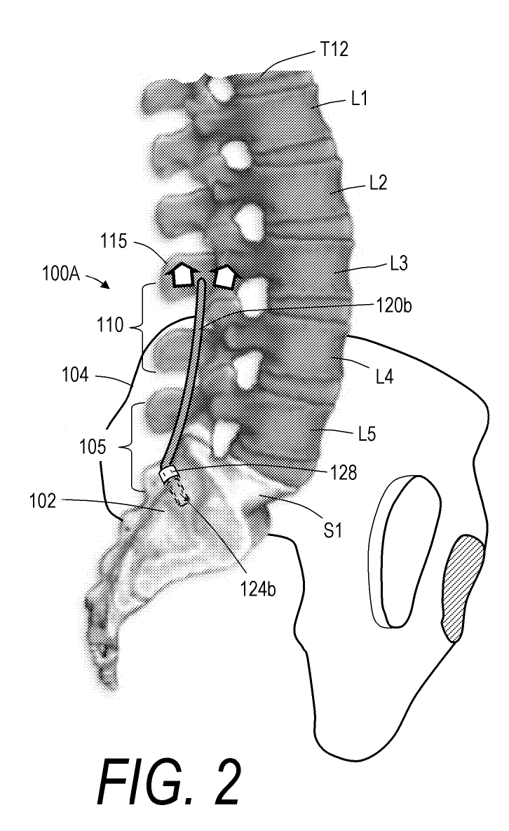

FIG. 1 shows a posterior view of one embodiment of a spine implant device 100A together with a patient's lumbar spine and portion of thoracic spine (T12-L5), sacrum 102 and ilium 104. FIG. 2 illustrates a side view of the spine implant device 100A. The implant device 100A has a first end portion or inferior portion 105 that can be fixed to the patient's sacral or iliac bones, which provide a platform for stabilizing a lumbar spine segment or a lower portion of the thoracic spine. The implant device 100A has a second end portion or superior portion 110 that can engage a spinous process 115 of a superior vertebra, for example from L1-L5 or a lower thoracic vertebra. In the illustrated embodiment, the spine implant device 100A includes paired extending elements or legs 120a and 120b (120 collectively) that extend superiorly to a cross element 122 (see FIG. 1) that can engage the spinous process 115.

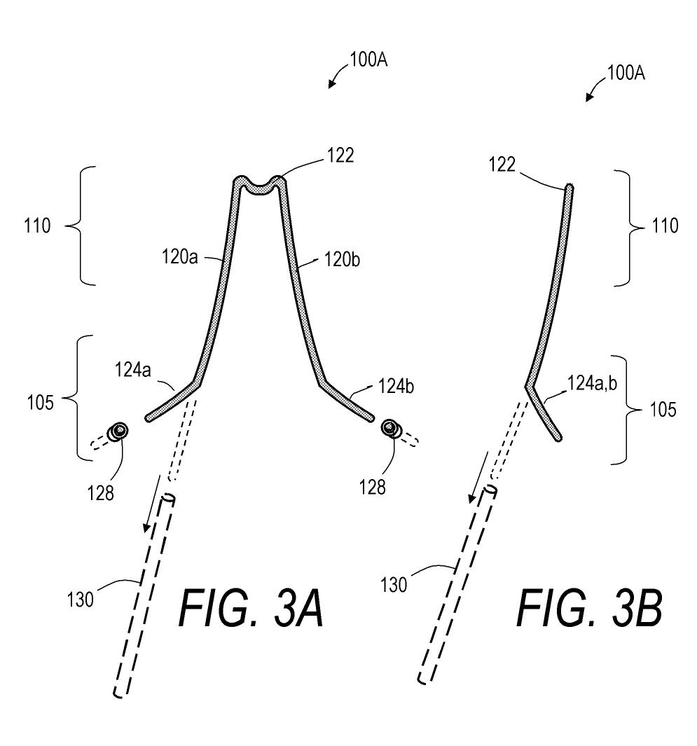

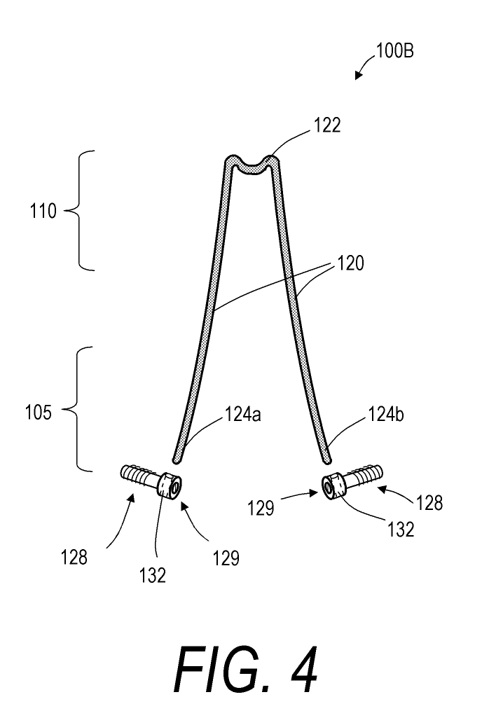

FIGS. 3A and 3B illustrate plan and side views of the spine implant device 100A of FIGS. 1 and 2, wherein paired extending elements 120a and 120b have proximal (inferior) ends 124a and 124b that in one embodiment can be inserted in an endosteal bore in the sacrum 102 or ilium 104. In one embodiment, the proximal (inferior) ends 124a and 124b can be inserted directly into a pathway in bone and can be cemented in place. In another embodiment, the proximal (inferior) ends 124a and 124b can be inserted in a central bore in paired bone screws, such as screws 128.

In a method according to one embodiment of implanting a spinal implant, the implant 100A can be introduced via two minimally invasively dissected tunnels on either side of the spine and through an incision in the region of the targeted spinous process 115 (e.g., L3 in FIGS. 1 and 2). The paired extending elements 120a and 120b can be of a shape memory alloy wire that can be maintained in a constraining sleeve 130, as shown in FIGS. 3A-3B. As illustrated in FIGS. 1 and 2, the spine segment can be stabilized and the tripod-like disc and facet joints can be off-loaded by the device 100A to carry loads. In one embodiment, the extending elements 120a and 120b and cross element 122 are of a resilient material. The extending elements 120a and 120b transmit a load to the sacral and/or iliac bones through the spinous process 115 to off-load at least one desired spine segment (e.g., in the direction of the arrow in FIG. 2).

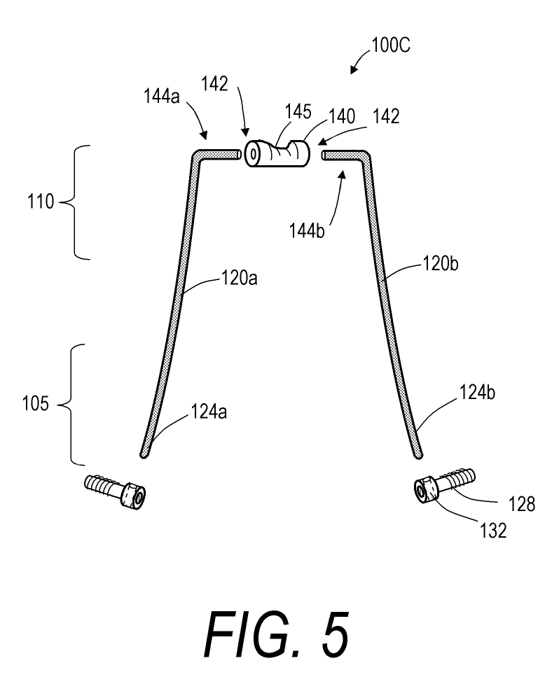

FIG. 4 illustrates another embodiment of an implant device 100B that is similar to the implant device 100A of FIGS. 1-3B, except that the paired extending elements 120a and 120b have proximal ends 124a and 124b that are generally co-linear with the extending elements 120a and 120b and can lockably couple with the heads 129 of the paired bone screws 128. In this embodiment, the ends 124a and 124b can be inserted through bores 132 in the heads 129 of the bone screws 128 and locked in place, for example, with set screws as is known in the art. It should be appreciated that any suitable rigid or flexible locking system known in the art of rods and pedicle screws can be used to couple the extending elements 120a and 120b to the bone screws 128 or other fasteners.

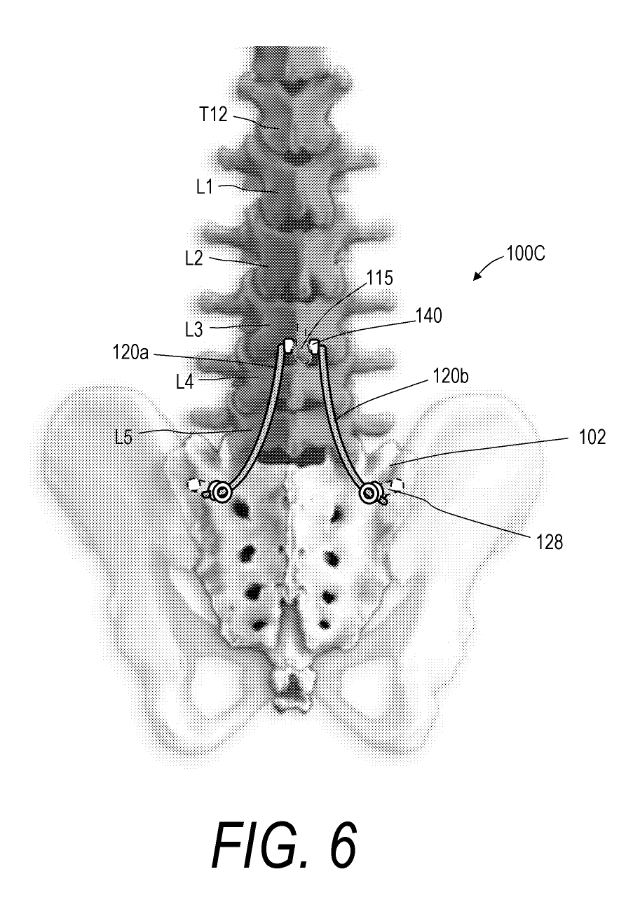

FIG. 5 illustrates another embodiment of an implant device 100C that is similar to that of FIGS. 1-4, except that the paired extending elements 120a and 120b are independent of each other and can be coupled together by a cross member 140 that can contact and support a spinous process. The cross member or connecting member 140 can have one or more bores 142 for receiving distal ends 144a and 144b of the extending elements 120a and 120b. Alternatively, the cross member 140 can have bores, such as those in bone screw 128, for lockably receiving the distal ends 144a and 144b of the extending elements 120a and 120b. The cross member 140 can have a saddle 145 for receiving a surface (e.g., inferior surface 115a as shown in FIG. 9) of the spinous process.

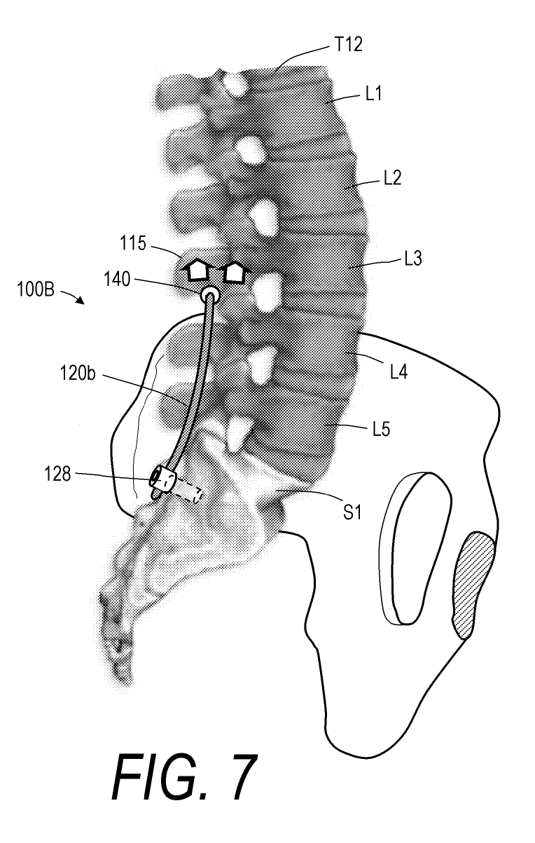

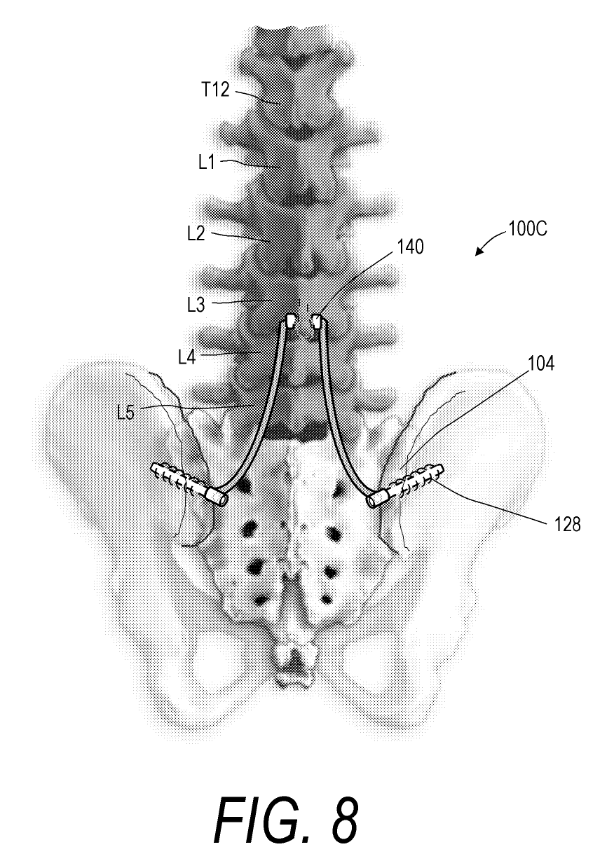

FIGS. 6 and 7 illustrate the implant device 100C of FIG. 5 implanted in a patient's spine to off-load and stabilize the spine segment. The bone screws 128 are fixed in the patient's sacrum 102. As shown in FIG. 7, the implant device 100C transmits a force in the direction of the arrows to off-load the spine segment.

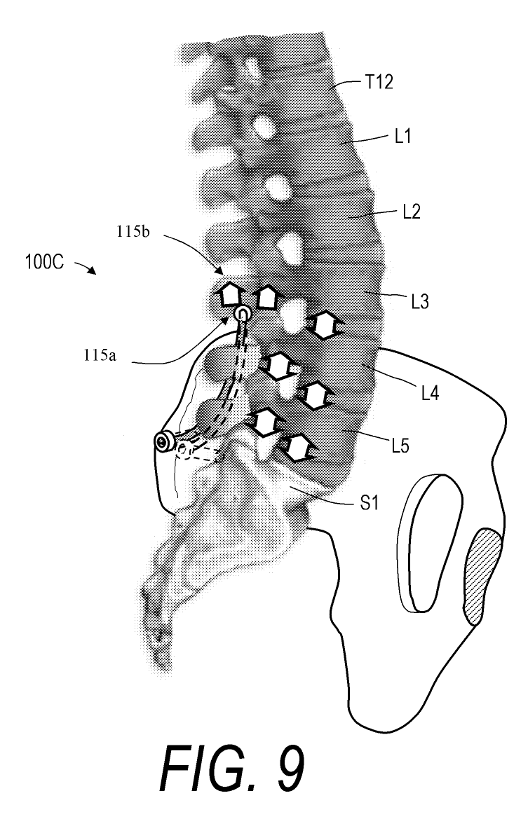

FIGS. 8 and 9 illustrate another implant device 100C, as in FIG. 5, with the inferior body portion 105 anchored by bone screws 128 to the patient's ilium 104. The bone screws 128 can be inserted at any suitable location in the posterior inferior iliac spine portion of the ilium 104. The bone screws 128 can be any suitable screw for use in surgical applications. In one embodiment, the bone screws 128 are self-tapping screws. In another embodiment (not shown), the bone (e.g., iliac or sacral) can be tapped in a minimally-invasive manner via small incisions in the patient's back prior to insertion of the bone screws in the tapped holes.

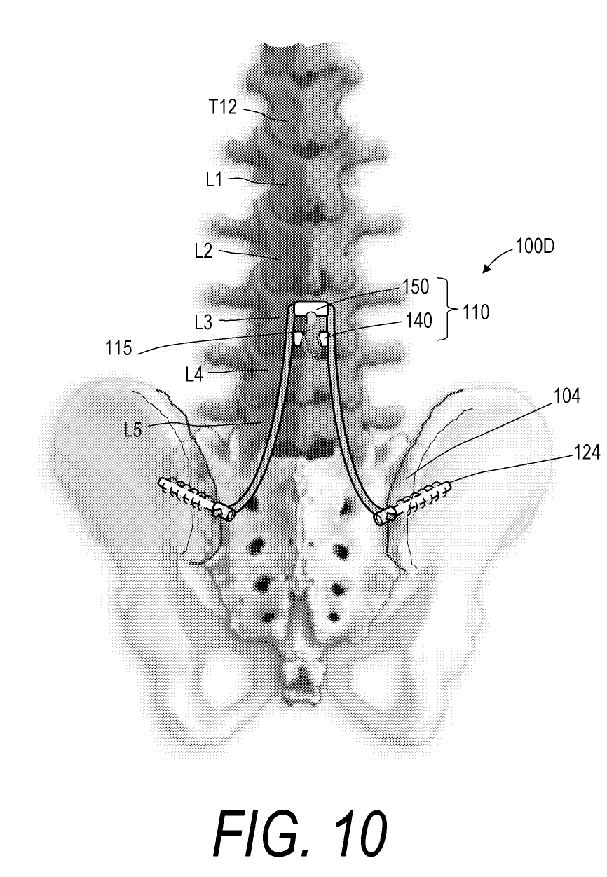

FIG. 10 illustrates another embodiment of an implant device 100D, which is similar to the implants illustrated in FIGS. 1-8, and has the inferior body portion 105 anchored by bone screws 128 to the patient's ilium 104. In the illustrated embodiment, the superior portion 110 of the implant 100D provides a second cross member 150 that can engage a superior surface 115b (see FIG. 9) of a spinous process 115. The implant device 100D can also include the cross member 140 to engage the inferior surface 115a of the spinous process 115. Thus, in the illustrated embodiment, the implant 100D can limit both flexion and extension of a spine segment.

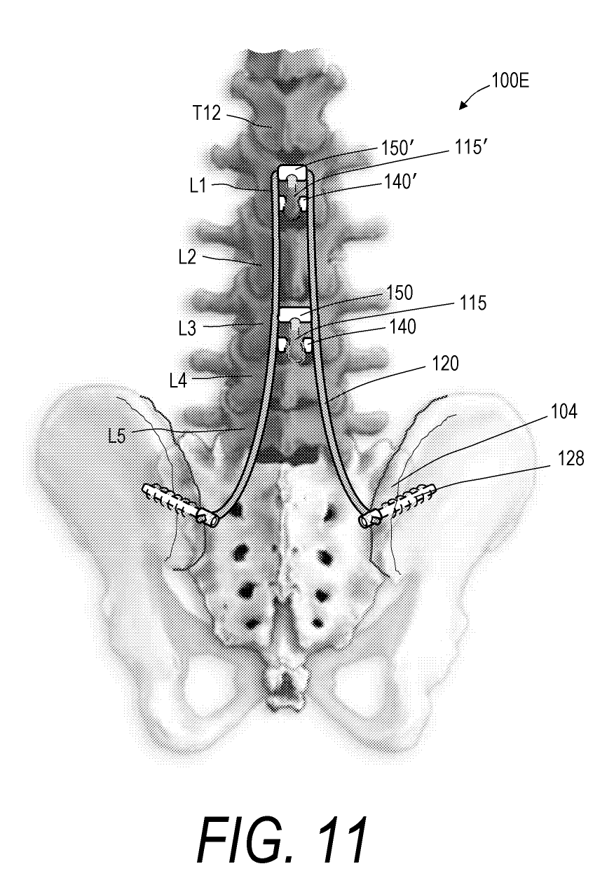

FIG. 11 illustrates another embodiment of an implant device 100E, which is similar to the implant 100D of FIG. 10, with the inferior body portion 105 again anchored by bone screws 128 to the patient's ilium 104. The superior portion 110 of the implant 100E provides a plurality of cross members 140, 150, 140′ and 150′ that can engage inferior 115a, and/or superior surfaces 115b (See FIG. 9) of multiple spinous processes 115, 115′. The engaged spinous processes 115, 115′ can be at adjacent levels or at spaced apart non-adjacent levels, as depicted in FIG. 11.

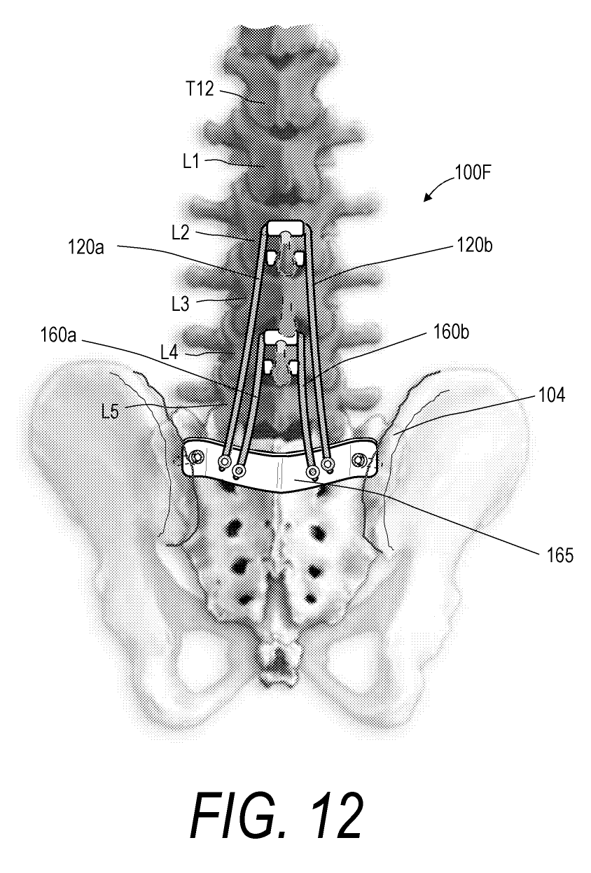

FIG. 12 illustrates another embodiment of an implant device 100F that is similar to the implant 100E of FIG. 11 in that it engages a plurality if different levels for stabilizing a spine segment, but the extending members 120a, b and 160a, b are independent of each other to carry loads to the sacrum or ilium. In the illustrated embodiment, the extending elements 120a, b and 160a, b are lockably and adjustably coupled to a flange 165 that is fixed to the sacrum and/or ilium.

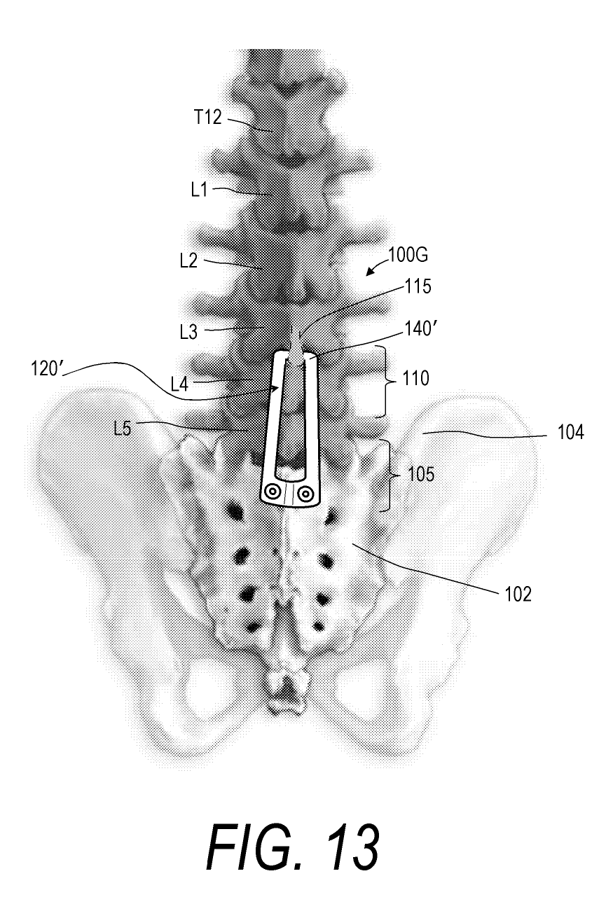

FIG. 13 illustrates another embodiment of an implant device 100G, similar to those described above, that has unitary plate-like member configured extension elements 120′ that extend to a cross element 140′ that can contact the inferior surface 115a of the spinous process 115.

In an alternative embodiment and method, the extension elements 120 of any embodiment disclosed herein can comprise a fluid expandable body that is filled with a flowable medium, which can comprise an in-situ polymerizable material. Further details on spinal implant devices, including implants with fluid expandable bodies, can be found in U.S. application Ser. No. 11/758,596 filed on Jun. 5, 2007 (Atty. Docket No. DFINE.014A), the entire contents of which are hereby incorporated by reference and should be considered a part of this specification.

Any implant body as depicted in FIGS. 1-13 can be fabricated of a metal, polymer, carbon fiber or composite to provide high stiffness and flexibility to act like a spring. That is, the implant body can include any suitable material that provides a desired spring constant, so that upon implantation as described above, the implant can exert a spring force onto the spinous process of the desired spine segment to off-load the spine segment. The mean cross section of any extending member can be from about 1 mm. to about 6 mm, and preferably between about 2 mm. and about 5 mm.

Any implant body as depicted in FIGS. 1-13 also can be fabricated with the extension elements 120 (see, e.g., FIG. 5) including a shock-absorber (e.g., pneumatic or hydraulic damping element) or spring-form such as a helical form, arcuate form or any compound curvature for functioning as a spring.

Certain embodiments described above provide new ranges of minimally invasive, reversible treatments that form a new category between traditional conservative therapies and the more invasive surgeries, such as fusion procedures or disc replacement procedures. One embodiment includes a system with implants configured for fixation in a sacrum or ilium with superior portions of the implant body engaging one or more spinous processes. The embodiments disclosed herein provide an MIS implant system for creating a spacing device that extends from a sacral-iliac platform to a targeted vertebra and off-loads the intervening discs and facet joints.

Certain embodiments include implant systems that can be implanted in a very minimally invasive procedure, and require only small bilateral incisions in a posterior approach. A posterior approach is highly advantageous for patient recovery. In some embodiment, the implant systems are “modular” in that separate implant components are used that can be implanted in a single surgery or in sequential surgical interventions. Certain embodiments of the inventive procedures are for the first time reversible, unlike fusion and disc replacement procedures. Additionally, embodiments of the invention include implant systems that can be partly or entirely removable. Further, in one embodiment, the system allows for in-situ adjustment requiring, for example, a needle-like penetration to access the implant.

In certain embodiments, the implant system can be considered for use far in advance of more invasive fusion or disc replacement procedures. In certain embodiments, the inventive system allows for dynamic stabilization of a spine segment in a manner that is comparable to complete disc replacement. Embodiments of the implant system are configured to improve on disc replacement in that it can augment vertebral spacing (e.g., disc height) and foraminal spacing at the same time as controllably reducing loads on facet joints—which complete disc replacement may not address. Certain embodiments of the implant systems are based on principles of a native spine segment by creating stability with a tripod load receiving arrangement. The implant arrangement thus supplements the spine's natural tripod load-bearing system (e.g., disc and two facet joints) and can re-distribute loads with the spine segment in spine torsion, extension, lateral bending and flexion.

Of particular interest, since the embodiments of implant systems are far less invasive than artificial discs and the like, the systems likely will allow for a rapid regulatory approval path when compared to the more invasive artificial disc procedures.

Other implant systems and methods within the spirit and scope of the invention can be used to increase intervertebral spacing, increase the volume of the spinal canal and off-load the facet joints to thereby reduce compression on nerves and vessels to alleviate pain associated therewith.

Although these inventions have been disclosed in the context of a certain preferred embodiments and examples, it will be understood by those skilled in the art that the present inventions extend beyond the specifically disclosed embodiments to other alternative embodiments and/or uses of the inventions and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above. Although particular embodiments of the present invention have been described above in detail, it will be understood that this description is merely for purposes of illustration. Specific features of the invention are shown in some drawings and not in others, and this is for convenience only and any feature may be combined with another in accordance with the invention. Further variations will be apparent to one skilled in the art in light of this disclosure and are intended to fall within the scope of the appended claims.

What is claimed is:

1. A spine treatment device, comprising:
an implant body extending from a first body portion to a second body portion, the first body portion comprising a pair of elongated extending members configured for fixation to a sacral or iliac bone of a spine, the second body portion comprising at least one connecting member extending transversely between the pair of extending members, the at least one connecting member configured to contact a surface of a spinous process of a spine segment to limit at least one of extension and flexion of the spine segment.

2. The device of claim 1, wherein the first body portion is removably coupleable to the second body portion.

3. The device of claim 1, wherein the first and second body portions are unitary.

4. The device of claim 1, wherein the implant body comprises two connecting members, at least one of the two connecting members extending between the pair of extending members.

5. The device of claim 1, wherein the connecting member comprises a saddle configured to contact a surface of the spinous process.

6. The device of claim 1, wherein the connecting member comprises a U-shaped surface configured to contact an inferior surface of the spinous process.

7. The device of claim 1, wherein the at least one connecting member is configured to contact at least one of an inferior surface and a superior surface of the spinous process.

8. The device of claim 1, further comprising at least one anchor element configured to anchor the implant body to the sacral or iliac bone.

9. The device of claim 8, wherein the anchor includes a threaded portion for threadably engaging the sacral or iliac bone.

10. The device of claim 8, wherein the anchor is a bone screw.

11. The device of claim 8, wherein the at least one anchor element comprises a pair of anchor elements that lockably couple to the extending members of the first body portion.

12. The device of claim 8, wherein the anchor element is a flange coupled to at least one of the sacrum and ilium.

13. The device of claim 1, wherein the extending members are plate-like members.

14. The device of claim 1, wherein the extending members are rod-shaped members.

15. The device of claim 1, wherein the extending members are configured to apply a spring force onto the spinous process to off-load the associated spine segment.

16. The device of claim 1, wherein at least a part of the first and second body portions comprise a shape memory alloy.

17. The device of claim 1, wherein the second body portion has a polymer surface coating.

18. An implant for treatment of a spine segment, comprising:
a pair legs coupleable to a sacral or iliac bone of a spine; and
first and second connecting members extending between the pair of legs and positionable on either side of a spinous process such that one of the connecting members contacts an inferior surface of the spinous process and another of the connecting members contacts a superior surface of the spinous process so as to limit extension and flexion of the spine segment.

19. The implant of claim 18, further comprising third and fourth connecting members extending between the pair of legs and positionable on either side of a second spinous process such that one of the third and fourth connecting members contacts an inferior surface of the second spinous process and another of the third and fourth connecting members contacts a superior surface of the second spinous process.

20. The implant of claim 19, wherein the spinous processes are of non-adjacent vertebrae.

21. A method for treating an abnormal spine segment, comprising:
fixating a first body portion of a stabilization device to at least one of a sacral bone and an iliac bone; and
positioning a second body portion of the stabilization device in contact with a spinous process of a vertebra, wherein the stabilization device varies a load-carrying characteristic of the spine segment.

22. The method of claim 21, wherein fixating comprises coupling the first body portion to bone screws coupled to at least one of the sacral bone and iliac bone.

23. The method of claim 21, wherein fixating comprises bi-laterally fixating the first body portion to the sacrum

24. The method of claim 21, wherein fixating comprises bi-laterally fixating the first body portion in the ilium

25. The method of claim 21, wherein positioning comprises positioning the second body portion in contact with at least one of an inferior surface and a superior surface of the spinous process to limit at least one of extension, flexion, rotation and lateral bending of the spine segment.

26. The method of claim 21, wherein positioning comprises positioning the second body portion in contact with the spinous process so as to limit extension of the spine segment.

27. The method of claim 21, wherein positioning comprises positioning the second body portion in contact with the spinous process so as to limit flexion of the spine segment.

28. The method of claim 21, further comprising coupling the second body portion to the first body portion.
